(19) United States
(12) Patent Application Publication
(10) Pub. No.: US 2024/0237993 A1
(43) Pub. Date: Jul. 18, 2024

(54) MEDICAL INSTRUMENT HAVING A CLEANING-OPTIMISED SPRING UNIT

(71) Applicant: Aesculap AG, Tuttlingen (DE)

(72) Inventor: Jan Dahmen, Seitingen-Oberflacht (DE)

(21) Appl. No.: 18/563,548

(22) PCT Filed: May 23, 2022
(86) PCT No.: PCT/EP2022/063927

§ 371 Date: Nov. 22, 2023

(30) Foreign Application Priority Data
May 26, 2021 (DE) 10 2021 113 559.1

(51) Int. Cl. A61B 17/16; A61B 17/00; A61B 17/28

(52) U.S. Cl. CPC A61B 17/1606; A61B 2017/00526; A61B 2017/2845

(57) ABSTRACT

A medical instrument includes two handle elements that are pivotable relative to one another and a spring unit that has two spring ends. Each spring end is connected to one of the handle elements so that when at least one of the handle elements is pivoted out of a starting position, the spring unit can pivot the handle element back into the starting position. The spring unit provides a substantially consistent spring force when the handle elements are pivoted. The spring unit includes a first spring leg and a second spring leg. At least one of the spring ends projects into a recess formed in the associated handle element and bonded to the associated handle element. The medical instrument can be produced by bending at least one of the spring legs, inserting the spring leg into an indentation in one of the handle elements, and bonding the spring leg to the handle element.

### CROSS-REFERENCE TO RELATED APPLICATION(S)

This application is the United States national stage entry of International Application No. PCT/EP2022/063927, filed on May 23, 2022, and claims priority to German Application No. 10 2021 113 559.1, filed on May 26, 2021. The contents of International Application No. PCT/EP2022/063927 and German Application No. 10 2021 113 559.1 are incorporated by reference herein in their entireties.

### FIELD

The present disclosure relates to a medical, in particular surgical, instrument, preferably a manually operable hand instrument, having at least two (a first and a second) gripping elements, wherein the gripping elements in particular have metal as material, are particularly preferably made of metal as material as a whole (for example stainless steel as material) and the gripping elements are pivotably mounted relative to each other via a bearing, in particular via a joint, and a spring unit/spring-elastic assembly which has two spring ends, which are each connected to one of the two gripping elements, so that when at least one of the two gripping elements pivots out of a base state (against an elastic force of the spring unit), pivoting back into the base state can be carried out/realized/achieved via the spring unit. The disclosure also relates to a method of manufacturing a medical instrument.

### BACKGROUND

In medical or surgical (hand) instruments, return springs are currently used that are configured as leaf springs and make contact over a large area with a branch or gripping element. Both the leaf spring and the gripping element have large, continuous surfaces facing each other which, starting from the common contact bearing surface, move continuously away from each other. It can also be said that the leaf spring and the gripping element have a semi-circular/arc-shaped contour with different radii in the portion of the fastening and successively increase the distance between them (from zero). While a partial surface of the leaf spring is in direct contact with a partial surface of the gripping element, the two surfaces move away from each other on the outside and form a very small gap between them, which gradually increases in size. As a result, it is very difficult to clean and sterilize the medical instruments in the area of the contact support surface. In addition, the area where the return spring and gripping element rest is exposed to contact corrosion or bimetal corrosion.

DE 10 2017 114 260 A1, for example, discloses a medical hand instrument in which a one-piece return spring is attached to a gripping element via a form fit. However, the disadvantage of this instrument is that elaborately milled receiving portions have to be provided on the branch or gripping element. In addition, a one-piece return spring is provided, so that a linearly increasing high spring force is created when the hand instrument is closed. This spring force counteracts a force to be applied by a user, such as a surgeon, in particular in a closed position, and thus reduces the maximum force that can be applied manually. Furthermore, the spring is subject to a high degree of deformation, so that there is an increased risk of the return spring breaking during continuous use.

### SUMMARY

The objects and objectives of the present disclosure are to avoid or at least reduce the disadvantages of the prior art and in particular to provide a medical, in particular surgical, instrument and a method for manufacturing a medical instrument which offers good cleanability, is simple and inexpensive to manufacture and at the same time maintains a constant force for pivoting the gripping elements over a large, in particular entire, travel range of the instrument. Furthermore, the instrument should in particular be small in size, have a simple design, be easy to handle and simplify maintenance.

The present disclosure thus relates to a medical instrument with two gripping elements (handlebars, handle levers, lever arms) which are pivotable relative to each other and a spring unit which is arranged between the gripping elements. The preferably U-shaped or V-shaped spring unit has two (spring) ends. Each of the two spring ends is attached to a corresponding/associated one of the two gripping elements, so that when at least one of the two gripping elements pivots from a base state, pivoting back into this base state is realized. This means that if one of the two gripping elements is pivoted manually by a user of the instrument, the spring unit can pivot the pivoted gripping element back into its base state as soon as the user releases the pivoted handle part. Preferably, the instrument has load arms opposite a hinge that pivotably connects the gripping elements. The medical instrument may also be configured in such a way that both gripping elements are pivoted when the instrument is used. If only one gripping element is intended to be pivoted, the other gripping element only serves as a counter support for the user's hand during pivoting. In particular, the medical instrument has a distal effective portion that is actuatable via the proximal gripping portion, such as a clamping portion (clamping/gripping branches) or cutting portion (cutting blades), etc.

According to the present disclosure, such a spring unit is thus provided in the instrument, which extends in particular from at least one of the two spring ends, particularly preferably from both spring ends, toward a bearing of the gripping elements, in particular a joint, particularly preferably a hinge, or a pivot axis, and is adapted to provide a constant/unvarying force/spring force/restoring force when the gripping elements pivot, in particular over an entire closing travel of the instrument (entire possible pivoting movement). Preferably, a spring unit having at least two parts is provided in the instrument to realize the unvarying spring force, which has at least a first spring portion, in particular a first spring leg, and a (separate) second spring portion, in particular a second spring leg, which are connected to each other, in particular coupled to each other in at least a partially movable manner. When the two legs or gripping elements are pressed together from the base state, a spring force of the spring unit (when pivoting into a closed position) can be maintained uniformly and homogeneously, in particular over the entire travel range of the closing action. There is no linear increase in the spring force. This special design supports a user of the instrument and ultimately also increases the potentially maximum closing force that can be applied and counteracts user fatigue.

In addition, the spring unit or the spring ends are connected to the gripping elements in a specially adapted manner. Specifically, at least one spring end is form-fittingly inserted into an associated geometric receiving portion/indentation formed on the gripping element and/or is firmly bonded to the associated gripping element. Inserting the spring end into the indentation causes the spring end to be geometrically fixed in at least one direction (and in particular in the opposite direction) by the form fit or undercut (and also in particular by rotation) relative to the gripping element. This locks at least one, in particular at least two possible degrees of freedom of movement (of a total of six degrees of freedom: three translational and three rotational) of the spring end relative to the associated gripping element, in particular in one direction of the longitudinal axis of the gripping element and in particular rotation about an axis of rotation perpendicular to the longitudinal handle axis and perpendicular to the longitudinal axis of the spring end.

This design enables a simple and cost-effective construction of the medical instrument, which is good and easy to clean and sterilize and meets the high requirements of a medical product. In particular, the spring unit is attached to the gripping elements in a non-detachable/loss-proof manner. Since the spring unit in particular only comes into contact with the associated gripping element at the at least one spring end in all pivot positions, in particular in the base state, the defined connection point of the spring end and gripping element, in particular the receiving portion/indentation, enables an individually adapted geometric design. If the spring end is also firmly bonded to the gripping element in such a way that a closed surface is formed in the connection area, good cleaning is ensured. With an indentation in particular, at least one of the two spring ends can protrude into an indentation and be firmly bonded to the gripping element so that a gap that forms between the indentation and the spring end is hermetically sealed toward an instrument environment in order to prevent the formation of germs.

In other words, the spring unit, in particular a spring leg, and at least one gripping element are adapted so that a spring end can be inserted into the gripping element or branch and/or is firmly bonded to it, in particular in such a way that no dirt can penetrate into or adhere to the interface, for example in a gap. In particular, at least one of the two spring legs is inserted into the branch and is furthermore firmly bonded in such a way that there is a hermetically sealed surface without undercuts at the connection point. When the spring end is inserted into an associated indentation, buckling is also prevented.

The spring unit and the gripping elements can thus be configured separately and manufactured cost-effectively and are securely connected to each other during mounting.

In particular, a contact point/connection point of the two spring ends can also be configured to be separable, in particular without tools, in order to ensure easy and effective cleaning or replacement of the spring unit.

In still other words, at least one of the two spring ends protrudes (form-fittingly) into a corresponding, in particular complementary indentation (as receiving portion) formed in the associated gripping element and which in particular forms at least one undercut in the distal direction along a longitudinal handle axis and/or is firmly bonded to the associated gripping element, in particular in such a way that a gap between indentation and spring end is hermetically sealed toward an instrument environment.

The term base state defines a position of the pivotable gripping elements in relation to each other, into which they move without external (manual) force, only due to the spring unit. In particular, this base state may be a (geometrically determined) maximum opening position of the instrument.

Preferably, the spring unit extends from at least one spring end toward a pivot axis or a bearing of the medical instrument, in particular distally. In particular, at least one spring extends from its spring end toward the pivot axis or the bearing, preferably distally. In other words, the spring unit, in particular the at least two springs, may be located between the two pivotable gripping elements and extend along these gripping elements toward a pivot axis or a bearing, in particular a hinge. The spring unit or the springs therefore do not protrude proximally or away from the bearing. This allows even better utilization of the installation space and further improves ergonomics. The surgeon does not have to be careful that the spring unit inadvertently gets caught on external objects such as a surgical drape or that it accidentally damages external objects such as tissue. The spring unit is located in particular between the gripping elements or is arranged between the gripping elements (at a similar height to a longitudinal axis of the instrument) and is in a sense enclosed by the gripping elements and protects the spring unit from the (lateral) outside by the gripping elements.

In particular, the indentation may be provided on the inside of the associated gripping element (i.e. opposite the other gripping element) and/or a longitudinal axis of the indentation may point toward the opposite gripping element. Since the indentation is open laterally inward and the spring end is inserted into it, the spring unit is held in a loss-proof manner and the spring force can be transmitted particularly well to the gripping element. The indentation is therefore not formed at the proximal end of the gripping element, but on the inside of the gripping element. In particular, this optimizes the installation space and arrangement of the spring unit in co-operation with the spring unit.

Advantageous embodiments are explained in particular below.

According to one aspect of the present disclosure, the at least one spring end and the associated gripping element may be welded and/or soldered together, in particular brazed with an additive, and/or glued, in particular at a contact region/contact boundary between an outer surface of the spring end and an internal surface of the associated gripping element, which is opposite the other gripping element. These firmly bonded connections are durable, easy to create and, in particular in the case of a circumferential weld seam or soldered seam, represent a boundary that is in particular hermetically sealed from the environment, into which germs cannot penetrate and which can also be sterilized even at high temperatures.

According to a further aspect of the present disclosure, at least one of the two spring legs may have a cantilever angle between the, in particular protruding, spring end and the associated gripping element (distal of the spring end seen in the longitudinal axis of the branch) of at least 30°, preferably of at least 45°, most preferably of at least 70° and in particular exactly 90°. It can also be said that the cantilever angle defines an angle between a longitudinal axis of the gripping elements and a longitudinal axis of the spring ends. This minimum cantilever angle ensures that a gap is formed with a sufficiently obtuse angle that can be cleaned easily.

In particular, a dimension from an end face of the spring end along a rectilinear portion of the spring end is at least 2 mm, preferably at least 5 mm, particularly preferably at least 10 mm, in order to realize a minimum gap height. In particular, a gap forming between a spring leg and the associated gripping element has a minimum height of 1 mm, preferably 3 mm, particularly preferably 5 mm. Preferably, a distance in the direction perpendicular to the longitudinal axis of the gripping element between an internal surface of the handle at the spring end and a facing surface of the spring unit after a proximal bend and/or at a point at a length of the spring leg of 15% of the total length (measured from the spring end), in at least the base state, is at least 1 mm, preferably 3 mm and particularly preferably 5 mm. This allows the instrument to be cleaned easily.

Preferably, at least one spring leg may be configured as a leaf spring or spring steel wire and, in particular, have the material X20CR13 or, according to DIN EN 10088, the material 1.4021, as the material or may be made entirely of this material. If the spring leg or the entire spring unit is made of this material, very good mechanical properties, very good polishability, good forgeability coupled with good chemical resistance and satisfactory weldability are provided.

According to one embodiment, the spring unit may be configured in two parts in the form of two leaf springs or two spring steel wires, which are connected/coupled to each other via a distal spring coupling, in particular via a distal fork-nose connection or a distal sphere-pan connection or a distal Y-t connection. Leaf springs are simple and inexpensive to manufacture, can be easily and irreversibly bent into the desired shape and offer reliable spring forces. Spring steel wires, on the other hand, are also inexpensive and meet the requirements for a mechanical property for corresponding forming. In particular, their circular cross-section means that they have no edges, making them particularly easy to clean. In the two-part form with the distal spring coupling, the spring forces remain constant over the entire closing path of the instrument or when the gripping elements pivot against each other in any pivoting position. A linear increase in spring force, as is the case with a one-piece spring unit, is avoided.

According to a further embodiment, in the case of an at least two-part spring unit, at least one of the two spring legs, in particular both spring legs, may be S-shaped with, in particular, a first proximal bending radius and/or a second opposing distal bending radius, wherein preferably a curve angle/bending angle between the tangential portions of the proximal bending radius (or of the partial circle segment) is 90° and/or the distal bending radius is 45°. The S-shaped design supports a spring-elastic force and geometric change or approximation of the spring leg with respect to the gripping element, in particular in such a way that the spring leg does not come into contact or abutment with the gripping element in any pivoted position, or at least not in the base state of the gripping elements with respect to each other.

In particular, the indentation may be a blind hole or a groove or a slit, in particular a passage slit. A spring steel wire with (approximately) the same diameter (press fit or clearance fit; similar to a pin in a bore) may be inserted into a blind hole with a defined diameter. If a leaf spring is used as a spring leg, the leaf-shaped/plate-shaped spring end (with a relatively smaller height than width) can be inserted into an elongated groove. If it is a continuous groove, for example, the leaf spring may also be inserted laterally into the groove instead of being inserted along its longitudinal axis. The groove may also have an undercut in the direction of insertion and the spring end may have an associated projection so that, when inserted laterally, a form-fit is formed in the direction of the longitudinal axis of the spring end. In addition, the indentation may have a depth (in the direction of the longitudinal axis of the protruding spring ends) of at least 2 mm and/or a maximum of 6 mm. The minimum depth ensures sufficient mechanical load-bearing capacity of the spring leg or spring unit.

According to a further aspect, the spring unit may be adapted with respect to the gripping elements in such a way that the first and/or second spring leg only comes into contact with the associated gripping elements at the spring end as a connection point in at least the base state, in particular in all positions in which the gripping elements are pivoted with respect to each other. This prevents abrasion and improves the durability of both the spring unit and the gripping elements.

Preferably, the spring unit may have a distal spring coupling that is mounted so that it can be detachably displaced and/or pivoted relative to each other.

Preferably, the indentation and/or the spring end is angular or oval (and not circular), so that a degree of rotation about the longitudinal axis of the spring end is also blocked. In particular, the spring end and indentation have such a shape that only a single geometric degree of freedom remains in only one direction, which is further preferably acted upon by a pre-tensioning force, for example due to the spring unit, in order to hold the spring unit in a loss-proof manner. Alternatively or additionally, the spring end may also or even be firmly bonded to the gripping element.

Preferably, the medical instrument may be configured as a multi-jointed branched forceps. In particular, the medical instrument may be configured as multi-action, in particular double-action, forceps, particularly preferably as double-action rongeurs. The spring structure disclosed herein is particularly suitable for this type of instrument. Such an instrument type of multi-action or double-action forceps, in particular rongeurs, has several joints, which are particularly stable. As a result, however, the joins also have a higher total internal friction (several joins result in a larger friction surface), which requires a higher restoring force of the spring (unit). This is different from the requirements for a return spring of neurological scissors or forceps. With the instrument type of double-action forceps, it is particularly disadvantageous if the spring force increases during the closing process, as the spring force is already very high at the start of the closing process. This makes the manual force required by the user when closing the double-action forceps a particularly great challenge, in particular since the double-action rongeurs are used to cut off pieces of bone, an operation which in itself requires considerable force. It is particularly advantageous for these double-action rongeurs that the indentation(s) or the receiving portion(s) in the gripping element(s) run at an angle toward a bearing, in particular a joint, or a pivot axis, as such a configuration reliably prevents the spring ends from slipping out under high spring force. In contrast, vertical receiving portions or indentations, i.e. receiving portions that extend perpendicular to a longitudinal axis of the associated gripping element, or even those that extend toward a user, are particularly susceptible to being pushed out of the indentation or receiving portion when high force is applied, rendering the instrument unusable and also jeopardizing the safety of a patient.

The objects and objectives of the disclosure are solved with respect to a method for manufacturing a medical instrument with a spring unit, in particular an instrument of the present disclosure, by the steps, in particular in this order: bending at least one spring leg, in particular a leaf spring or a spring steel wire; forming, in particular imprinting and/or grinding, a distal portion of the spring leg, preferably distal grinding of a rounding; inserting the at least one spring leg into an indentation formed in a gripping element, in particular in the form of a blind hole or a groove; preferably orienting the spring leg relative to the associated gripping element; firmly bonded connecting, in particular welding and/or soldering, the spring leg to the associated gripping element. These steps of the method provide an assembly of a medical instrument with at least one gripping element and one spring leg, which is easy and inexpensive to manufacture and easy to clean. Production, including simple mounting, is easy to carry out and also inexpensive.

Preferably, after the step of firmly bonded connecting, the method may comprise the step of tempering the spring leg with the associated gripping element and/or the step of brushing and/or sandblasting the spring leg with the associated gripping element.

Furthermore, the method may include a step of an overall assembly of the instrument.

In other words, in particular a medical instrument can be provided in which preferably two leaf springs or two spring wires/spring steel wires, one for each branch (gripping element), are inserted and/or welded and/or soldered and/or glued into blind holes or grooves in the branches. Since the springs do not rest against the branches anywhere except at their connection part (in at least the base state), good cleaning or cleanability is guaranteed. The two-part spring, which in spring concepts are in distal contact via spring force, a fork-nose connection or a sphere-pan connection, has the advantage that the spring force does not increase linearly when the medical instrument is closed, particularly in the form of forceps. As a result, the closing forces remain virtually constant.

### BRIEF DESCRIPTION OF THE DRAWINGS

The present disclosure is explained in more detail below based on preferred embodiments with reference to the accompanying Figures. The following is shown:

FIG. 1 shows a plan view of a medical instrument of a first preferred embodiment, in which the spring unit is form-fitted and firmly bonded to the handles;

FIG. 2 shows a detailed view of a distal spring coupling of the two leaf springs of the spring unit of the medical instrument from FIG. 1;

FIG. 3 shows a top view of a medical instrument of a further, second preferred embodiment, in which the spring unit has a distal Y-t connection;

FIG. 4 shows a detailed view of a distal connection of the spring unit of a further preferred embodiment of an instrument in the form of a sphere-pan connection;

FIG. 5 shows a top view of a spring unit of a further preferred embodiment of a medical instrument;

FIG. 6 shows a top view of a medical instrument of a preferred embodiment, in which the spring unit of FIG. 5 is inserted;

FIG. 7 shows a detailed view of the distal spring coupling of the spring unit of FIG. 6; and

FIG. 8 shows a flow chart of a process for manufacturing a medical instrument according to a preferred embodiment.

The Figures are schematic in nature and are intended only to aid understanding of the present disclosure. Identical elements are provided with the same reference signs. The features of the various embodiments may be interchanged. Any disclosure related to the method according to the present disclosure also applies to the medical instrument of the present disclosure, just as any disclosure related to the medical instrument also applies to the method according to the present disclosure.

### DETAILED DESCRIPTION

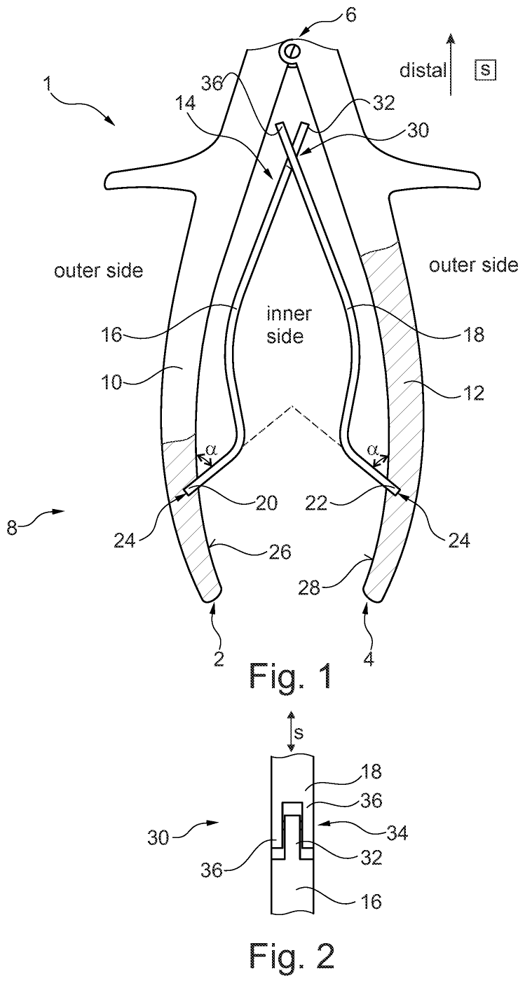

FIG. 1 shows a partial view of a medical instrument 1 of a first preferred embodiment in a base state. The instrument 1, in the form of a hand instrument, is configured as branched forceps of the forceps type. For this purpose, the instrument 1 has two levers 2, 4, which are pivotably connected to each other via a hinge 6. This allows the two levers 2, 4 to be pivoted in a pivot plane S in relation to each other, similar to a pair of scissors or grasping forceps. The proximal portions of the levers 2, 4 (proximal to the hinge 6 and pointing toward the user) form a gripping portion 8 with corresponding gripping elements or handles 10, 12, which are substantially symmetrical to each other with respect to a longitudinal instrument axis. The gripping portion 8 is also symmetrical to the pivot plane S.

A two-part spring unit 14 in the form of a two-part leaf spring is provided between the first handle 10 and the second handle 12, which form a first spring leg 16 and a second spring leg 18 of the spring unit 14 and which are releasably coupled to each other at their respective distal ends and are displaceable and pivotable.

The respective free spring ends 20, 22 of the spring legs 16, 18 are located in an indentation in the form of a groove formed in the respective handles 10, 12, which is formed on the inside of the handles 10, 12 on opposite internal surfaces 26, 28 projecting into the handles 10, 12. The indentations 24 are rectilinear and symmetrical to each other with respect to a longitudinal instrument axis and point (seen in plan view) slightly obliquely forward in a distal direction. The spring ends 20, 22 protrude into the indentations 24 and thus form a cantilever angle α between the distal outer side of the leaf spring (spring end portion), i.e. the surface that points distally, and the internal surface 26 or 28. This also substantially corresponds to an angle between a longitudinal branch axis or longitudinal handle axis along the handle 10, 12 and the spring end 20, 22 or a spring end portion adjoining the spring end 20, 22 or a spring end longitudinal axis.

The spring ends 20, 22 are also firmly bonded, i.e. welded, to the internal surfaces 26, 28, so that a gap between the indentations 24 and the respective spring ends is hermetically sealed toward an instrument environment. This ensures that, on the one hand, the spring unit 14 is securely attached to the instrument 1 or connected to it and, on the other hand, that gaps and other volumes that are difficult to access and in which germs could settle are prevented.

This special design of the instrument 1 with both form-fitting and firmly bonded connection of spring unit 14 and handles 10, 12 and also the special cantilevered portion of the spring ends 20, 22 from the handles 10, 12, enables cost-effective manufacture of the instrument 1, a high mechanical load capacity, a long service life and also exceptionally good cleanability, in particular sterilizability, since in particular a sufficiently large distance between the spring legs 16, 18 and the handles is also ensured. Since a cantilever angle α of at least approx. 45° is selected, a sufficiently obtuse angle is formed between spring end 20, 22 and handle 10, 12, which also promotes good cleaning. In particular, the form fit with indentation 24 and associated spring end 10, 12 ensures sufficient mechanical force of the spring unit 14 so that the instrument 1 is durable and functioning is guaranteed even after a very high number of swivel movements. Tearing out is prevented. In particular, the weld seam ensures sealing against the environment and also increases the mechanical strength of the connection.

The two leaf springs are made of material 1.4021 in accordance with DIN EN 10088, which provides very good mechanical properties, good polishability coupled with good chemical resistance and weldability.

FIG. 2 shows in detail a distal spring coupling 30 of the instrument 1 in FIG. 1 in the form of a distal fork-nose connection, viewed in the direction from distal to proximal. The free distal end of the first spring leg 16 has a nose 32, which is enclosed between a fork 34 with two prongs or arms 36 or side walls in a longitudinally displaceable and pivotable manner guided in the longitudinal instrument axis. In this way, the distal spring coupling 30 can follow a movement when the instrument 1 pivots and the spring force remains constant over the entire pivot path. Mounting is also simplified. The two distal ends are detachably coupled to each other in that, due to their pre-tensioning force, nose 32 and fork 34 are engaged and pressed elastically against each other. To a certain extent, a spring-pre-tensioned hinge is created at the distal end as a spring coupling 30.

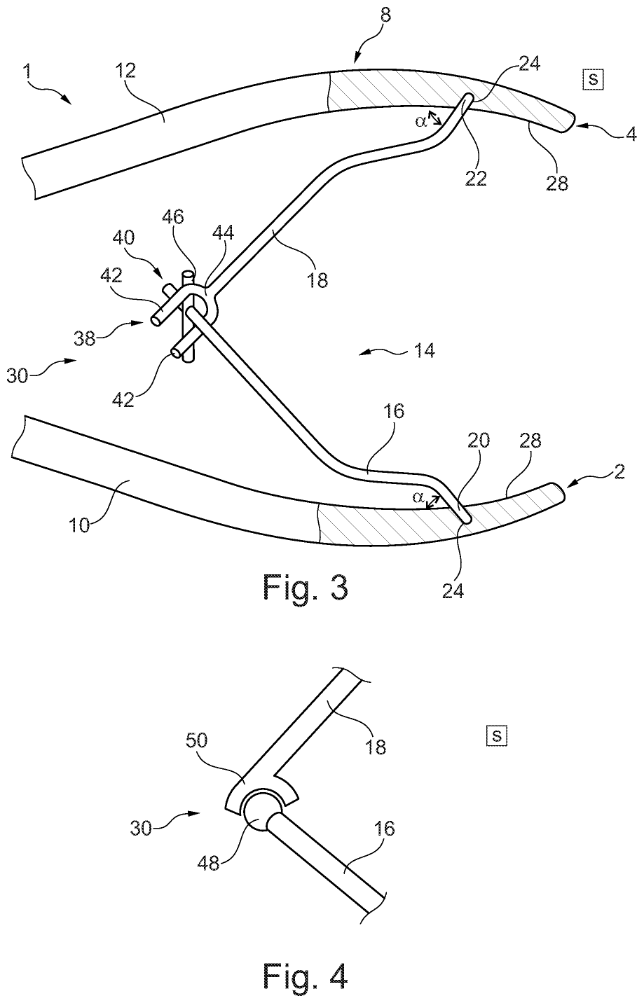

FIG. 3 shows a medical instrument 1 of a further, second preferred embodiment. In contrast to the first embodiment, the spring unit 14 and the associated indentations 24 are configured differently. In particular, the two spring legs 16, 18 are not configured as leaf springs but as an S-shaped spring steel wire, which is inserted into an indentation 24 configured as a blind hole. In addition, the distal coupling 30 is configured as a Y-t connection having a Y-shaped distal end portion/Y-portion 38, which is interlocked with a t-shaped, distal end portion t-portion 40 and is detachably in contact and coupled. The Y portion 38, like the entire second spring leg 18, has a circular cross-section in its two parallel Y arms 42. The Y arms 42 converge at a curved bifurcation 44. The t portion 40 has a crosspiece 46 with a circular cross-section that is perpendicular to a longitudinal axis of the first spring leg 16, so that a t-shaped or X-shaped portion is formed to a certain extent, which is in contact with the Y portion in an interlocked manner. Both spring legs 16, 18 are pre-tensioned against each other. In particular, in this embodiment of FIG. 3, the crosspieces 46 are arranged distally with respect to the two Y arms 42, so that the crosspieces 46 form a kind of stop for the Y portion 38 in the distal direction. In other words, the spring leg 16 is accommodated proximally of the two crosspieces 46 between the two Y arms 42. Due to the pre-tensioning force, in particular when the two handles 10, 12 are pressed together, the Y portion 38 is pressed distally, but is blocked by the two crosspieces 46 extending transversely to the spring leg 16. On the other hand, the t portion 40 is held in the fork of the Y portion 38 so that the two spring legs 16, 18 are detachably coupled to each other and provide a constant spring force over the entire pivoting movement path.

This embodiment with a Y-t connection enables good elastic bending of the two spring legs 16, 18, which are made of spring steel wire and are bent in an S-shape (irreversibly) or have such a shape along their longitudinal axis, while at the same time providing a secure (detachable) connection at the distal spring coupling 30. This design is particularly cost-effective to manufacture.

FIG. 4 shows a detailed view of a distal portion of the spring unit 14, here the spring coupling 30 of a further preferred embodiment. The distal spring coupling 30 in FIG. 4 can be used in the instrument 1 instead of the Y-t connection in FIG. 3. In contrast to the spring coupling 30 of the second embodiment in FIG. 3, the spring coupling of FIG. 4 has a sphere-pan connection with a distal sphere 48 formed on the first spring leg 16 and a pan 50 formed on the second spring leg 18 instead of a Y-t connection. Specifically, the sphere 48 is spring-pre-tensioned in a shell-shaped receiving portion of the pan 50 and a detachable connection or coupling (against the pre-tension of the spring unit 14) is created between the distal ends. This special coupling means that when the handles 10, 12 are pivoted out of a base state, the two spring legs 16, 18 are initially pushed distally due to the freedom of movement and deform elastically. However, since a (sphere) joint has been created by the sphere 48 and the pan 50, the distal ends can move and pivot toward each other so that a spring force remains uniform during a pivoting movement, such as a closing movement in a closing direction, or a virtually constant spring force is achieved.

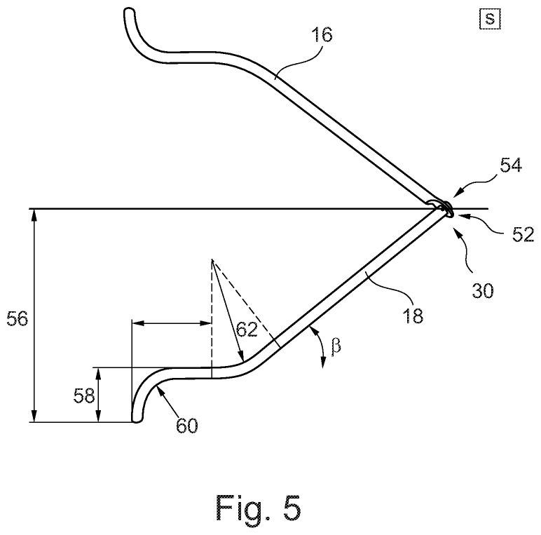

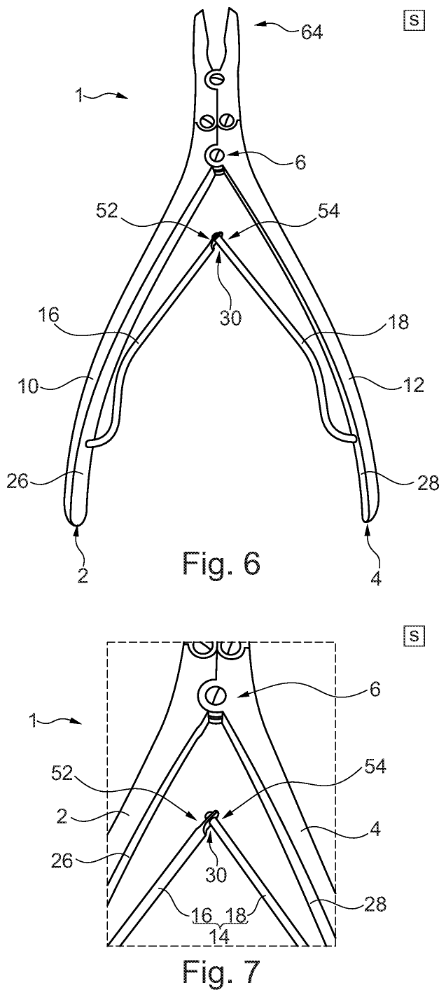

FIG. 5 shows a spring unit 14 according to a further preferred design, which is not yet connected to the handles (and is not yet pre-tensioned). The spring legs 16, 18 are symmetrical to each other except for the distal end. The proximal spring ends 20, 22 have a bend with a first proximal bending radius 60 (in particular a radius R6), which runs through 90° in a right-hand bend (seen in FIG. 5 at the second spring leg 18). After a short rectilinear portion (in particular over a length of 9 mm), a left-hand bend (as seen in FIG. 5; second spring leg 18) follows around a bending angle β of 45° with a larger distal bending radius 62 (approximately or exactly three times as large a bending radius, in particular R17). This is followed by a straight portion, the distal end portion of which makes a slight left turn (in particular by about 5°) and has a spherical rounding at the end.

The first spring leg 16 is S-shaped symmetrically to the second spring leg 18, but has a pan-shaped imprint 52 at its distal end, the pan/shell-shaped receiving portion of which is open toward the second spring leg 18 or toward the distal spherical rounding. The spherical distal end of the second spring leg 18 is inserted into this receiving portion. A maximum dimension 56 in pivot plane S in the direction perpendicular to the longitudinal instrument axis in the non-pretensioned state is in particular approximately 1.3 times a maximum dimension in pivot plane S in the direction of the longitudinal instrument axis. A dimension 58 of a cantilevered portion of the spring end 20 in the pivot plane S in the direction perpendicular to a longitudinal instrument axis is approximately 20% of the maximum dimension 56. In particular, a dimension 58 of the cantilevered portion is 11 mm. In particular, the spring end 20, 22 has a rectilinear portion which extends over approximately one third or one half of the dimension 58 of the cantilevered portion, in particular over 5 mm.

FIG. 6 and FIG. 7 show a top view and a detailed top view in the area of the distal spring coupling 30 of a medical instrument 1 according to a further preferred embodiment. The medical instrument 1 is configured as multi-jointed branched forceps and has a distal active section 64 in the form of forceps. The spring unit 14 of FIG. 5 is inserted in this instrument. Specifically, the two spring ends 20, 22 protrude at the internal surfaces 26, 28 into indentations (not shown) of the handles 10, 12.

A cantilever angle α is greater than in the first embodiment and is approximately 80°. The proximal bending radius 60 adjoins the spring end 20, 22 in each case. As a result, a defined gap is provided on the inside of the handles 10, 12, which has a high minimum size and ensures good cleanability due to the roundings both due to the circular cross-section of the S-shaped spring steel wire and the bending radius 60. In addition, the instrument 1 is easy and inexpensive to manufacture.

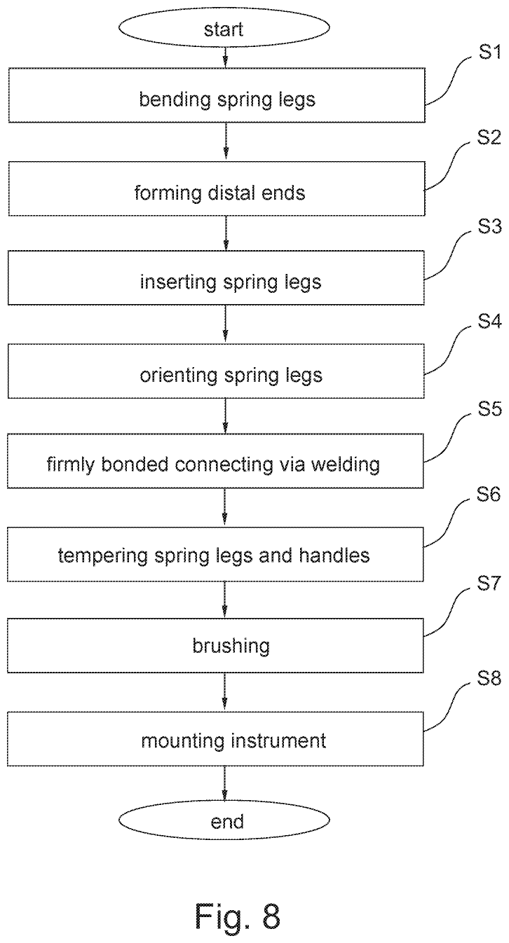

FIG. 8 shows a flowchart of a method for manufacturing an instrument 1 according to a preferred embodiment. In this embodiment, the method manufactures an instrument 1 according to the present disclosure.

In a first step S1, both a first and a second spring leg 16, 18 are bent into the correct shape. In particular, the spring leg 16, 18 is in the form of a spring steel wire or a leaf spring and is bent into an S-shape (according to a sketch).

In a step S2, the distal portions of the two spring legs 16, 18 are formed, which are later coupled together distally. Specifically, the free end of the first spring leg 16 is imprinted in such a way that a flat spoon-shaped or pan-shaped structure/pan 52 is formed, which forms a cup-shaped receiving portion. The other distal end of the second spring leg 18 is ground distally to obtain a spherical/spherical rounding 54.

The order of steps S1 and S2 is not decisive, so that these two steps may also be carried out in reverse order.

This is followed by step S3 of inserting the two spring legs 16, 18 into an indentation 24 formed in each of the handles 10, 12 in the form of a blind hole, so that a form-fitting connection is created between the spring legs 16, 18 and the associated handles 10, 12.

The two spring legs 16, 18 are then aligned with respect to the associated handles 10, 12 in a step S4. In particular, the distal spherical rounding may already be loosely inserted into the complementary pan 52 at this point and the two spring legs 16, 18 can be pre-tensioned against each other in order to check correct alignment.

In this aligned position, the spring legs are then firmly bonded together in step S5. In the present case, the spring leg is welded to the associated gripping element, in particular with filler. Specifically, a circumferential weld seam is formed on the handles 10, 12 around the spring ends 20, 22 or on the end portions, so that a gap that forms between the spring ends 20, 22 and the indentation 24 is hermetically sealed.

After step S5 of firmly bonded connecting, step S6 of tempering the spring legs 16, 18 with the associated gripping element 10, 12 takes place. In particular, the entire instrument 1 or all components are tempered.

In step S7, the handles 10, 12 and the spring unit 14 are brushed and sandblasted to create a smooth surface, particularly at the connection point of handle 10, 12 and spring legs 16, 18.

Finally, step S8 is followed by the overall assembly of instrument 1.

1. A medical instrument comprising:
a first gripping element;
a second gripping element; and
a spring unit,
the first gripping element and the second gripping element being made of a metal and pivotably mounted relative to each other via a bearing,
the spring unit comprising a first spring end and a second spring end, the first spring end being connected to the first gripping element, and the second spring end being connected to the second gripping element, so that when at least one of the first gripping element and the second gripping element pivots out of a base state, pivoting back into the base state is carried out via the spring unit,
the spring unit extending from at least one of the first spring end and the second spring end toward the bearing,
the spring unit adapted to provide a substantially constant spring force when the first gripping element and the second gripping element pivot, and
at least one of the first spring end and the second spring end protruding into an indentation formed in one of the first gripping element and the second gripping element and/or is firmly bonded to said one of the first gripping element and the second gripping element.

2. The medical instrument according to claim 1, said at least one of the first spring end and the second spring end and said one of the first gripping element and the second gripping element are at least one of:
welded together,
soldered together
glued.

3. The medical instrument according to claim 1, wherein said at least one of the first spring end and the second spring end comprises a cantilever angle between said at least one of the first spring end and the second spring end and said one of the first gripping element and the second gripping element of at least 30°.

4. The medical instrument according to claim 1, wherein the spring unit comprises a first spring leg and a second spring leg, and wherein at least one of the first spring leg and the second spring leg is configured as a leaf spring or spring steel wire.

5. The medical instrument according to claim 4, wherein each of the first spring leg and the second spring leg is a leaf spring or a spring steel wire, the leaf springs or the spring steel wires being detachably coupled to each other via a distal spring coupling.

6. The medical instrument according to claim 4, wherein at least one of the first spring leg and the second spring is S-shaped.

7. The medical instrument according to claim 1, wherein the indentation is a blind hole or a groove or a slit.

8. The medical instrument according to claim 1, wherein the spring unit is adapted with respect to the first gripping element and the second gripping element in such a way that the spring unit only comes into contact with the first gripping element and the second gripping element at the first spring end and the second spring end, respectively, as a connection point in at least the base state.

9. A method for manufacturing the medical instrument according to claim 1, the spring unit of the medical instrument comprising a first spring leg and a second spring leg, the method comprising the steps of:
bending at least one of the first spring leg and the second spring leg;
forming a distal portion or end of said at least one of the first spring leg and the second spring leg;
inserting said at least one of the first spring leg and the second spring leg into the indentation; and
connecting said at least one of the first spring leg and the second spring leg to said one of the first gripping element and the second gripping element.

10. The method according to claim 9, further comprising the step of tempering said at least one of the first spring leg and the second spring leg with said one of the first gripping element and the second gripping element and/or comprises the step of brushing and/or sandblasting said at least one of the first spring leg and the second spring leg with said one of the first gripping element and the second gripping element.
